(19) United States
(12) Patent Application Publication
(10) Pub. No.: US 2008/0091058 A1
(43) Pub. Date: Apr. 17, 2008

(54) URETHRAL SLING IMPLANTATION USING NEEDLE SHEATHS

(76) Inventors: Rodney W. Bosley, Chester Springs, PA (US); Marvin O. Andrews, Bloomington, IN (US); John H. Ward, Spencer, IN (US)

Correspondence Address: BRINKS HOFER GILSON & LIONE/CHICAGO/COOK, PO BOX 10395, CHICAGO, IL, 60610, US

(21) Appl. No.: 11/870,851

(22) Filed: Oct. 11, 2007

Related U.S. Application Data
(60) Provisional application No. 60/829,582, filed on Oct. 16, 2006.

(51) Int. Cl. A61F 2/02

(52) U.S. Cl. 600/37

(57) ABSTRACT

Instruments for implanting a urethral sling in female patients, and a better technique for implanting the sling, are disclosed. Porcine small intestine submucosa is processed into a sturdy yet assimilable sling for supporting a urethra of a female patient. The sling is implanted using ligature carriers and sheaths to minimize contact between the sling and the vaginal and abdominal tissues of the patient. The ligature carriers are implanted into the patient via a suprapubic or vaginal route (antegrade or retrograde). The sling is then attached to the ligature carriers and is drawn into the patient through the sheaths. Then sheaths are then removed and the sling is adjusted and trimmed.

### CROSS-REFERENCE TO RELATED APPLICATIONS

This application claims priority from U.S. Provisional Application No. 60/829,582, titled “Urethral Sling Implantation Using Needle Sheaths,” filed on Oct. 16, 2006, the entirety of which is fully incorporated by reference herein.

### FIELD OF THE INVENTION

The field of the invention is surgery, and in particular a method and an apparatus for correcting urinary incontinence.

### BACKGROUND OF THE INVENTION

Early procedures for the correction of urinary incontinence in females used sutures for elevating one or more portions of the urethra or urethrovesical junction. Some of these procedures used sutures only, as seen in U.S. Pat. No. 5,013,292. This patent discloses the use of a cannula and a needle for bringing sutures through incisions in the lower abdomen into the vagina, and then securing the sutures to support the urethra, or to support an insert or a sling for supporting the urethra. The procedures used, however, depend on an elaborate procedure and only a few threads to entirely support the affected areas. The method and equipment described herein have not been widely adopted.

U.S. Pat. No. 5,611,515, describes another earlier procedure. This procedure uses sutures and optionally, tissue staples, to anchor urethral supports to the pubic bone. The urethra is supported by sutures, or at most a small suture support structure that is centered on the urethra, but does not give much support beyond a very narrow area under the urethra. Suture passers may be used by surgeons to thread sutures to the area of interest.

Another technique is disclosed in U.S. Pat. No. 5,899,909. This patent discloses a sling implanted under the urethra, the sling made of a knitted polypropylene mesh. The sling may be enclosed in a thin polyethylene sheath, to protect the sling and to avoid having the rough edges of the netting irritate or damage body tissues. The mesh, made of polypropylene, may be rejected by the body. In addition, when the mesh is implanted, it is tightened at both ends. This may lead to complications and corrective surgery, even removal of the sling, may be required if the tension was too great. U.S. Pat. No. 6,491,703 is similar to U.S. Pat. No. 5,899,909, using a polypropylene mesh, and needles that are curved over about a quarter circle. In addition, there are handles with relatively complicated structures for attaching the mesh to the needles and to the handles.

U.S. Pat. No. 6,638,210 also discloses an apparatus and a method for placing a sling under a urethra of a female patient. In this patent, the needles, not the mesh, have sheaths for protecting the tape. In addition, the needles may be hollow and have a connector at one end for connecting to a source of a local anesthetic, e.g., a Luer lock connector for connecting to a syringe with lidocaine or other anesthetic. While helpful to the patient, this is an expensive needle. The other end of the needle has an eye so the surgeon can use a suture to attach the sling to the needle, for drawing the sling through the sheath. This may be a time-consuming way to attach the sling. What is needed is a better way to attach the sling to the needle. These and other advantages of the invention, as well as additional inventive features, will be apparent from the description of the invention provided herein.

### BRIEF SUMMARY OF THE INVENTION

One aspect of the invention is an assembly for placing a support sling under a urethra. The assembly includes a support sling comprising a central portion and a first and a second end portion, at least one ligature carrier comprising a needle with a curved portion and also including a hook at a distal portion of the needle for grasping an end portion of the sling. The assembly also includes at least one sheath suitable for partially enclosing the at least one ligature carrier, and a handle for manipulating the at least one ligature carrier, wherein the sheath is configured to accept and hold the at least one ligature carrier and to allow passage of the ligature carrier and the sling through the sheath.

Another aspect of the invention is an assembly for implanting a support sling under a urethra. The assembly includes two ligature carriers, each ligature carrier comprising a needle with a first curved portion and also comprising a hook at a distal portion of the needle, and a support sling comprising a central portion and two support portions, each support portion also comprising an aperture for receiving the hook. The assembly also includes two sheaths suitable for at least partially enclosing the ligature carriers, and at least one handle for manipulating the ligature carriers, wherein each sheath is configured to accept and hold the ligature carrier and to allow passage of the ligature carrier and the sling through the sheath.

Another aspect of the invention is a method for implanting a sling. The method includes placing each of two ligature carriers into a sheath, and implanting the sheaths into a patient so that the hooks of the ligature carriers extend beyond the sheaths and through a vaginal incision of the patient, on each side of a urethra of the patient. The method also includes inserting the hooks into the apertures of the support sling, drawing the sling into one of the sheaths and then the other of the sheaths so as to minimize contact between the sling and tissues of the patient and to place the sling under the urethra and between the urethra and a vaginal wall of the patient. The method also includes removing one of the sheaths and then the other of the sheaths; and adjusting the sling within the patient.

Yet another aspect of the invention is a method of implanting a urethral sling. The method includes steps of placing a first ligature carrier into a first sheath and placing a second ligature carrier into a second sheath, the ligature carriers each comprising a hook at a distal end of the ligature carrier, and creating a first pathway and a second pathway, the pathways extending from a vagina of a patient, under a urethra of the patient, to supporting tissue above the urethra of the patient. The method also includes placing the first ligature carrier and first sheath into the patient and the second ligature carrier and second sheath into the patient, attaching a sling to the hooks of the ligature carriers to apertures in the sling, drawing the sling into the first sheath and the second sheath using the needles, removing the sheaths from the pathways, and adjusting the sling.

Other embodiments and advantages of the invention will become more apparent from the following detailed description when taken in conjunction with the accompanying drawings.

### BRIEF DESCRIPTION OF THE DRAWINGS

The accompanying drawings incorporated in and forming a part of the specification illustrate several aspects of the present invention and, together with the description, serve to explain the principles of the invention. The drawings are meant to be illustrative rather than limiting. In the drawings:

FIGS. 1a and 1b depict a STRATASIS® sling useful in embodiments of the present invention;

FIG. 2 depicts a handle and needle useful with the sling of FIG. 1;

FIG. 3 depicts a closer view of the a handle of FIG. 2;

FIG. 4 depicts a sheath useful with the handle and needle of FIG. 2;

FIG. 5 depicts a cross section of the sheath of FIG. 4;

FIGS. 6a-6b depict cross sections of alternative sheath embodiments;

FIGS. 7-8 depict alternative distal ends of needles; and

FIG. 9 is a flowchart depicting a method of using a kit with the sling, needles, and sheaths as described herein.

While the invention will be described in connection with certain preferred embodiments, there is no intent to limit it to those embodiments. The intent is to cover all alternatives, modifications and equivalents as included within the spirit and scope of the invention as defined by the appended claims.

### DETAILED DESCRIPTION OF THE PRESENTLY PREFERRED EMBODIMENTS

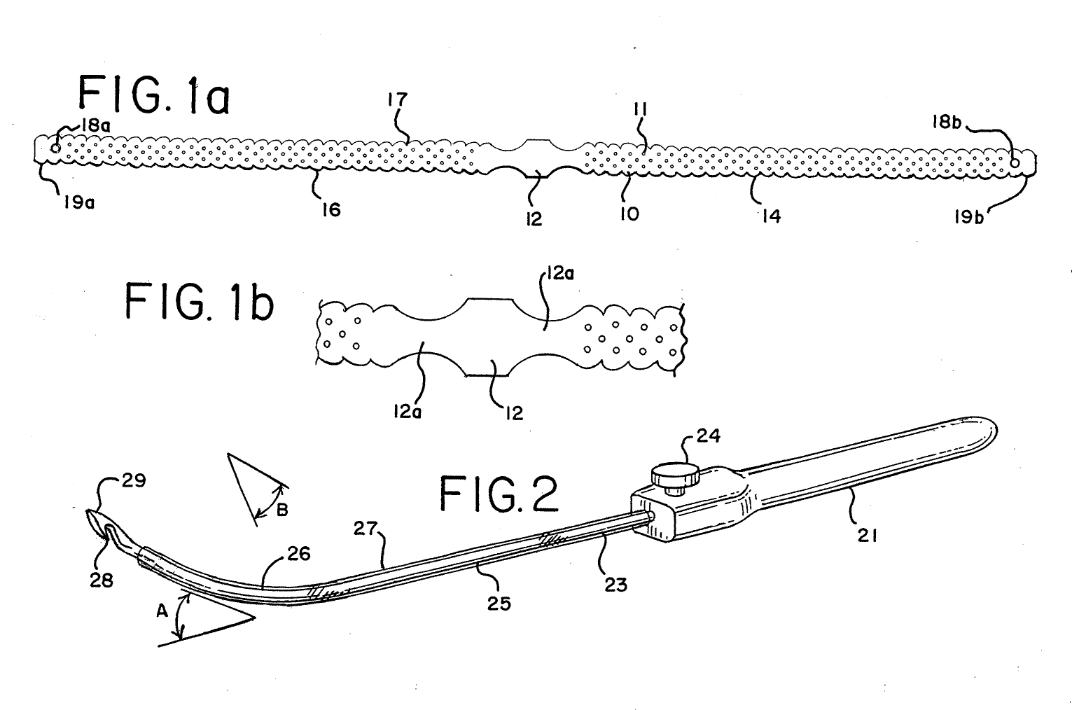

Embodiments of the present invention preferably use a sling made from porcine small intestine submucosa (SIS), although other slings or supports for the urethra may also be used. One sling useful in kit embodiments is depicted in FIGS. 1a and 1b. Sling 10 includes a central portion 12 and two tails 14, 16, preferably with serrations as shown, to better anchor the sling to tissues of the patient. Each tail has an aperture 18a, 18b in an end tab 19a, 19b of the tail for direct attachment to a needle. In order to make end tabs 19a, 19b a little longer, for easier grasping by the surgeon, apertures 18a, 18b may be placed a little further from the ends, say about 2-3 cm, rather than only 1 cm margin. Central portion 12 connects to the tails through transition portions 12a. Central portion 12 is preferably, but not necessarily, about 1.5 to about 2.0 cm long and about 1.5 to about 2.0 cm wide. Each tail is about 19-20 cm long, and transitions 12a are gently radiused so that the narrowest portion of the sling is no less than about 0.6 to about 1.2 cm wide. The sling preferably also has a plurality of perforations 11 to promote ingrowth into body tissues when the sling is implanted between the patient's urethra and vaginal wall.

Embodiments of the slings preferably include features for ameliorating potential causes of pull-out or roll-over of a sling, or a combination of pull-out and roll-over. In some cases, the problem of roll-over may be related to the support portion of the sling, since the primary consideration of this portion in most cases is to provide support for about 1-2 cm of the urethra, spreading the mechanical load and avoiding a stress concentration whereby the sling could damage the urethra. At least one contributor to roll-over forces and roll-over may be the endopelvic fascia, running near the urethra and roughly perpendicular to the sling. The sling may be implanted near the fascia, where the bulk and stiffness of the endopelvic fascia may interfere with a flat, planar installation of the sling by compressing the sling where the fascia pass through the sling. The sling may then be rolled or bunched into a narrow band under the urethra, depriving the urethra of broader, less stressful support.

To address this problem, the sling provides relief features near the support portion of the sling. The relief features provide relief for any endopelvic fascia passing transversely to the orientation of the sling when implanted. Narrowing transitions for providing relief are preferably gently radiused so that the sling continues to provide maximum support for the urethra. The transitions are preferably narrower than the adjacent support portion and the left or right side “tail” or supporting portion of the sling. In addition to or as an alternative to narrower portions, relief may be effectively provided by modifying any material in the relief area to render it less resistant to deformation or displacement by surrounding patient tissues. For example, material in the relief area may be windowed, sliced, perforated or otherwise sufficiently weakened or otherwise modified to facilitate the passage of endopelvic fascia without curling or rolling the intermediate portion of the sling device.

Other features may be added to the sling to increase resistance to pull out, for example anchors that provide convoluted surfaces, surfaces that interfere with movement of the sling, thus increasing resistance to pullout or movement. The features or anchors may be protuberances such as barbs, whereby the sling is provided with a plurality of small anchors to prevent movement once the sling is placed into a patient. In addition, or instead of barbs or protuberances, the sling may be provided with serrations on its edges, as shown in FIG. 1, in order to resist pullout. Other features that may help in anchoring the sling include perforations in the sling, in the support portions or in the transition regions, or both. Some of these anchoring features, especially penetrations or perforations, may also help to encourage the ingrowth of bodily tissue into the sling, thus supporting and stabilizing the sling. These features may help to stabilize the sling within the patient by increasing the surface area that opposes movement within the patient's body.

Processes for making multi-layer laminates from a plurality of individual layers of material are disclosed in U.S. Pat. Nos. 5,885,619 and 5,711,969, hereby incorporated by reference in their entirety. Additional teachings as to processing and layering the materials are disclosed in U.S. Pat. Nos. 5,733,337, 5,995,110, 5,993,844, 5,997,575, and 6,206,931, all of which are hereby incorporated by reference in their entirety. These patents teach the harvesting, cleansing, sterilizing and processing the submucosa from a variety of sources, including the alimentary, respiratory, urinary or genital tracts from bovine, ovine and porcine sources. Included in the processing are processes to dehydrate and compress the submucosal tissues. Processes may include vacuum drying, dead-weight pressing or other pressing, lyophilizing, room temperature or heat pressing, perforating, layering, and so on. Slings made from submucosal tissues by these and other processes are meant to be used in the present invention.

The multiple-layer embodiments may contain two laminate to eight or even more laminate of collagenous material. A preferred embodiment is a four-layer or an eight-layer material made from porcine SIS, and vacuum pressed to bind the layer in the thickness dimension. Any numbers of methods may be used to produce laminates, including the processing described above, mechanical reinforcements in the third (thickness) dimension, and the like. One preferred embodiment is a central portion having eight layers and arms or tails having four layers. The central portion may be prepared using a vacuum pressing process, resulting in a product that is relatively clear or translucent, i.e., allowing some light to pass through but in which objects on the other side are not at all clear or easily discernable. The arms may be prepared by a lyophilizing process, resulting in arms that are opaque, i.e., allowing the passage of no or almost no light. In preferred embodiments, the thickness of the central portion with eight layers is about the same thickness as the four-layer arms.

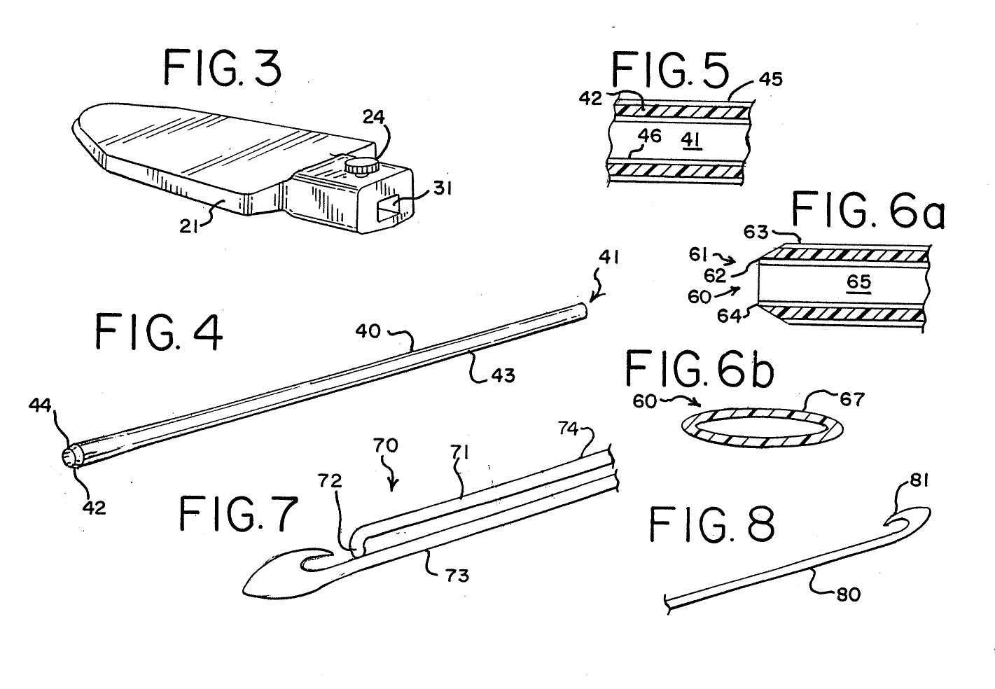

FIGS. 2 and 3 depict a preferred needle and handle to be used to implant the sling. The handle 21 includes an aperture 31 and a set screw 24 for securing the needle 23 into the handle. Needle 23 is preferably a modified Stamey-type needle with a curve 26 at about 70 degrees, angle A, to the longitudinal axis of the proximal portion 25 of the needle. The distal portion 29 of the needle includes a hook 28 with a back-angle, angle B, that is about 135 degrees to about 150 degrees to the longitudinal axis of distal portion 29. The hook is preferably at a gentle approach, with no sharp surfaces, so that the physician may hook sling end tab 19 using aperture 18a or 18b, without tearing or damage to the sling. The needle 23 is covered with a thin, flexible sheath 27.

The needle is preferably made from stainless steel or any other medically-acceptable material, and preferably has a uniform diameter of about 2-3 mm, although other diameters may be used. The distal and proximal ends of the needle may be flattened for easier insertion into handle 21, and for ease of attachment of hook 28 into an aperture of a sling. The handle may be made from stainless steel or from molded plastic. The ligature carriers or needles and the sheaths may be inserted via a suprapubic route, from incisions in the patient's lower abdomen to one or more incisions in the patient's vagina. Alternately, the needles and sheaths may be inserted via a vaginal route, that is, from one or more incisions in the patient's vagina, upwards on either side of the urethra, to incisions in the patient's lower abdomen.

A thin, flexible sheath for use in the kit is depicted in FIGS. 4-5. Tubular sheath 40 includes a central lumen 41, wall 42 and an outer surface 43. As shown in the cross-sectional view of FIG. 5, the outer surface may have a first coating 45 and the inner surface may have a second coating 46. The sheath is preferably about 15 to 20 cm long, a little shorter than needle 23. The sheath is preferably made from polyurethane, a polyurethane blend, a fluoro-polymer, or other PTFE-type material. The sheath may have a slight bevel 44 for easier insertion into the patient, and the sheath may also be slightly tapered, so that one end of the sheath is slightly wider than the other end.

At the end of the procedure, when the sheath is removed from the patient, the sheath may be extracted beginning with the wider end, and will pull out a little more easily if there is a slight taper. About 1-2 degrees of taper is sufficient; more or less may be used. The sheath should have a slightly larger diameter than the needle. In one embodiment, the sheath is about 10 Fr diameter (about 3.33 mm, 0.131 inches) and the needle is about 9 Fr diameter (about 3 mm, about 0.118 inches). This provides a clearance on each side between the needle and sheath of about 0.15 mm or about 0.006 inches.

In addition to the taper, the sheath may be coated on its inside, its outside, or both. The coating 46 on the inside will be in contact with the sling as the sling is pulled through the sheath. The coating may be a thin layer of polyethylene or fluoro-polymer, or it may be a lubricious coating, such as a hydrophilic coating, to ease the passage of the sling and the needle as they are drawn through the sheath. The coating on the outside preferably makes the sheath very lubricious as the sheath is inserted into the patient. This coating may be a lubricious coating, such as a hydrophilic coating, or it may simply be a very thin, slick coating of PTFE-type material, or polyethylene. The outer coating may also include a local anesthetic, such as lidocaine or bupivacaine, to ease the sensation of the sheath and needle being implanted into the patient. There are many ways to coat sheaths, needles or other objects with a small, desired, and controlled amount of an anesthetic.

In one embodiment, the sheath may be tapered on at least one end, the distal end, for ease of insertion into the patient. FIG. 6a depicts a sheath 60 having a beveled surface. In this embodiment, sheath 60 has a beveled entrance 61. Sheath 60 is made from a polymeric material 62 and may include inner and outer coatings 63, 64, as well as a central lumen 65 to allow passage of the needle and sling. In another alternative embodiment, as shown in FIG. 6b, sheath 67 may have a generally ovate or elliptical cross-section. The aspect ratio, the ratio of the major diameter to the minor diameter, may be from about two to about ten, because the width of the urethral sling is typically much greater than its thickness. Other ratios, from greater aspect ratios to circular cross-sections, may also be used.

Alternative embodiments of the needles or ligature carriers are depicted in FIGS. 7-8. In FIG. 7, the distal end 70 of a ligature carrier 74 captures the sling between two arms 71, 73 of the distal end. The arms may include a hook or transverse portion 72 at an angle, such as a 90° angle as shown. Arms 71, 73 may have different stiffnesses or thicknesses, or they may be the same. The arms are manufactured or treated so that they have natural tendency to remain closed. In practice, the surgeon may extend the needles from the suprapubic region of the patients, through the tunnels created, until the needles emerge from the vaginal incision. Arms 71, 73 are spread to engage an aperture from a urethral sling, and are then allowed to close, capturing the sling. The surgeon then pulls the sling into place. Another needle embodiment is depicted in FIG. 8, in which ligature carrier 80 has a distal end with a 180° hook for capturing the sling before implantation.

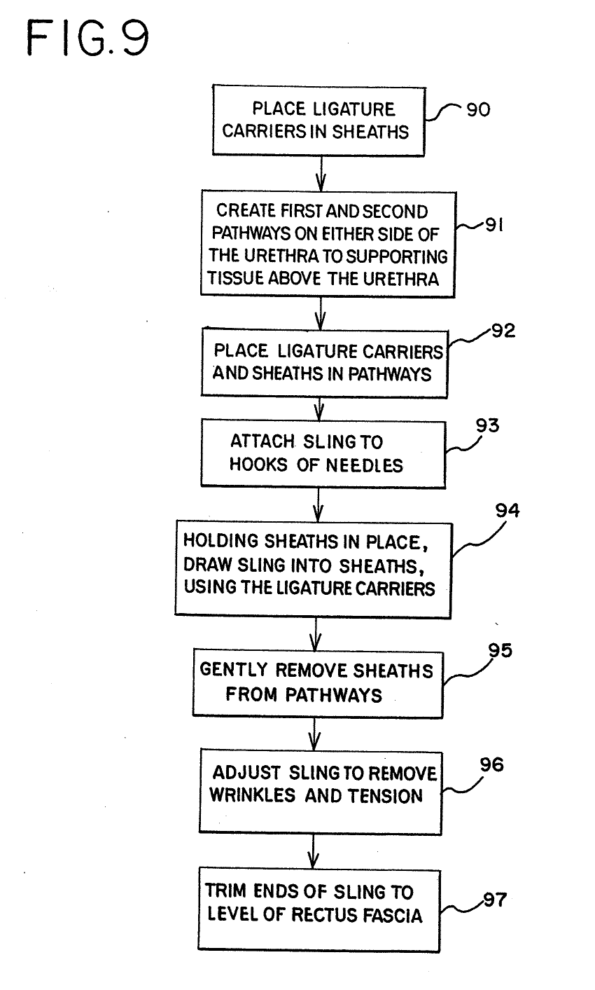

A physician using the ligature carriers, sheaths, and a sling as described above, may implant the sling in a patient, as described in the flowchart of FIG. 9. The ligature carriers, such as modified Stamey needles with a curve near the distal end, are placed 90 into sheaths, the sheaths preferably made from a lubricious material, such as a polytetrafluoroethylene material or a polyethylene material. The physician then creates a pathway 91 on either side of the patient, the pathway extending from under the urethra to supporting tissues above the urethra of the patient, such as to a suprapubic region of the patient. The physician then places 92 the ligature carriers and sheaths in the pathways, and attaches 93 the sling to the hooks of the needles.

Holding the sheaths in place, preferably from a suprapubic position, the physician then draws 94 the sling into the sheaths using the ligature carriers, on both sides of the patient. After this step, the physician gently removes 95 the sheaths from the pathways, leaving the sling in place. The physician then adjust 96 the sling to remove wrinkles and tension from the sling. The physician should then trim 97 the ends of the sling, using a sling cutter, to the level of the rectus fascia. The sling cutter is described in co-pending application Ser. No. 11/399,781 titled “Percutaneous and Endoscopic Cutters,” filed on Apr. 7, 2006, which is assigned to the assignee of this patent. The abdominal and vaginal incisions are then closed.

This procedure is preferably accomplished with an antegrade placement technique, although a retrograde technique may also be used. In this technique, an appropriate amount of anesthesia may be used, and two small (0.5 to 1.0 cm) abdominal skin incisions are made, one on each side of the midline, and just above the pubic symphysis. A vaginal incision is made approximately 1 cm from the outer urethral meatus, a midline vertical anterior vaginal incision of approximately 2 cm toward the bladder neck. A small paraurethral space is made by dissecting the vaginal wall from the periurethral fascia, which are preferably left adjacent the urethra and as intact as possible.

In the antegrade placement technique, the ligature carriers extend through the sheaths and each is brought from one of the abdominal skin incisions, along the posterior of the pubic bone, and guided through the vaginal incision. Once the ligature carriers and sheaths are in place, the procedure described above is accomplished. A similar technique is followed for the retrograde placement method.

All references, including publications, patent applications, and patents cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) is to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context.

The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments are described herein, including the best mode known to the inventors for carrying out the invention. Variations of those preferred embodiments may become apparent to those of ordinary skill in the art upon reading the foregoing description. Skilled artisans will use such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. An assembly for placing a support sling under a urethra, the assembly comprising:
a support sling comprising a central portion and a first and a second end portion;
at least one ligature carrier comprising a needle with a curved portion and also comprising a hook at a distal portion of the needle for grasping an end portion of the sling;
at least one sheath suitable for partially enclosing the at least one ligature carrier; and
a handle for manipulating the at least one ligature carrier, wherein the sheath is configured to accept and hold the at least one ligature carrier and to allow passage of the ligature carrier and the sling through the sheath.

2. The assembly of claim 1, wherein the support sling also comprises transitions between the central portion and the end portions.

3. The assembly of claim 1, further comprising an aperture in the first end portion and the second end portion, each aperture configured for receiving the hook.

4. The assembly of claim 1, wherein the at least one ligature carrier is a Stamey needle modified to include the hook.

5. The assembly of claim 1, wherein a cross section of the sheath comprises an aspect ratio of at least 2:1.

6. The assembly of claim 1, wherein the hook is formed at an angle of about 135 to about 150 degrees to a longitudinal axis of the distal portion of the needle.

7. The assembly of claim 1, wherein the sling comprises extracellular material from small intestine submucosa.

8. An assembly for implanting a support sling under a urethra, the assembly comprising:
two ligature carriers, each ligature carrier comprising a needle with a first curved portion and also comprising a hook at a distal portion of the needle;
a support sling comprising a central portion and two support portions, each support portion also comprising an aperture for receiving the hook;
two sheaths suitable for at least partially enclosing the ligature carriers; and
at least one handle for manipulating the ligature carriers,
wherein each sheath is configured to accept and hold the ligature carrier and to allow passage of the ligature carrier and a portion of the sling through the sheath.

9. The assembly of claim 8, wherein the ligature carriers are made from stainless steel.

10. The assembly of claim 8, wherein the hook is formed at an angle of about 135 degrees to 150 degrees to a longitudinal axis of the distal portion of the needle.

11. The assembly of claim 8, wherein the sling comprises arms that are 20 opaque.

12. The assembly of claim 8, wherein the central portion is relatively translucent.

13. The assembly of claim 8, wherein an aspect ratio of the sheaths is at least 2.5.

14. The assembly of claim 8, wherein at least one of the ligature carriers or the sheaths further comprises a coating.

15. The assembly of claim 8, wherein at least one of the ligature carriers or the sheaths further comprises a lubricious coating or an anesthetic.

16. A method of using the assembly of claim 8, the method comprising:
placing each ligature carrier into a sheath;
implanting the sheaths into a patient so that the hooks extend beyond the sheaths and through a vaginal incision of the patient, on each side of a urethra of the patient;
inserting the hooks into the apertures of the support sling;
drawing the sling into one of the sheaths and then the other of the sheaths so as to minimize contact between the sling and tissues of the patient and to place the sling under the urethra and between the urethra and a vaginal wall of the patient;
removing one of the sheaths and then the other of the sheaths; and
adjusting the sling within the patient.

17. A method of implanting a urethral sling, the method comprising:
placing a first ligature carrier into a first sheath and placing a second ligature carrier into a second sheath, the ligature carriers each comprising a hook at a distal end of the ligature carrier;
creating a first pathway and a second pathway, the pathways extending from a vagina of a patient, under a urethra of the patient, to supporting tissue above the urethra of the patient;
placing the first ligature carrier and first sheath into the patient and the second ligature carrier and second sheath into the patient;
attaching a sling to the hooks of the ligature carriers to apertures in the sling;
drawing the sling into the first sheath and the second sheath using the needles;
removing the sheaths from the pathways; and adjusting the sling.

18. The method of claim 17, wherein the pathways are created by placing the ligature carriers and sheaths into the patient.

19. The method of claim 17, wherein the ligature carriers and sheaths are inserted via a suprapubic route.

20. The method of claim 17, wherein the ligature carriers and sheaths are inserted via a vaginal route.
